(19) United States
(12) Patent Application Publication
(10) Pub. No.: US 2005/0047975 A1
(43) Pub. Date: Mar. 3, 2005

(54) UV sterilisation air-flow chamber

(75) Inventors: Wai Choi Tang, Hong Kong SAR (CN); Wilson Sai Mo Wong, Hong Kong SAR (CN)

Correspondence Address: ALIX YALE & RISTAS LLP, 750 MAIN STREET, SUITE 1400, HARTFORD, CT, 06103, US

(73) Assignee: Winsource Industries Limited

(21) Appl. No.: 10/911,008

(22) Filed: Aug. 4, 2004

(30) Foreign Application Priority Data
Aug. 26, 2003 (HK) 03106108.3

(51) Int. Cl. A61L 9/18

(52) U.S. Cl. 422/121

(57) ABSTRACT

A sterilisation chamber includes an elongate housing, an air inlet port adjacent to one end of the housing, an air outlet port adjacent to the other end of the housing, and an elongate ultraviolet fluorescent tube mounted within the housing and extending from the inlet port to the outlet port. Air passing through the housing from one end to the other encounters ultraviolet light emitted from the fluorescent tube en route from the inlet port to the outlet port.

### BACKGROUND OF THE INVENTION

The present invention relates to the sterilisation of air. More particularly, although not exclusively, the invention relates to the sterilisation of air that might be delivered to or from a patient in an infectious disease ward of a hospital.

Infectious airborne diseases are readily transmitted from person-to-person in environments such as hospital infectious disease wards. Patients are often placed in isolated rooms or cordoned-off areas and infected air extracted from such areas should be disinfected before spreading to neighbouring areas where others might otherwise contract airborne diseases.

### OBJECT OF THE INVENTION

It is an object of the present invention to overcome or substantially ameliorate the above disadvantage and/or more generally to provide a UV sterilisation air-flow chamber through which air drawn off from a space is disinfected en route to being exhausted to atmosphere or a neighbouring area.

### DISCLOSURE OF THE INVENTION

There is disclosed herein a sterilisation chamber comprising:
an elongate housing,
an air inlet port at or adjacent to one end of the housing,
an air outlet port at or adjacent to the other end of the housing, and
an elongate ultraviolet fluorescent tube mounted within the housing and extending substantially from the inlet port to the outlet port such that air passing through the housing from one end to the other encounters ultraviolet light emitted by the fluorescent tube en route from the inlet port to the outlet port.

Preferably, the chamber further comprises another elongate ultraviolet fluorescent tube parallel to and coextensive with the first said tube.

Preferably the chamber has attached to its air inlet port and/or air outlet port an air pump to cause air flow through the chamber from the inlet port to the outlet port.

### BRIEF DESCRIPTION OF THE DRAWINGS

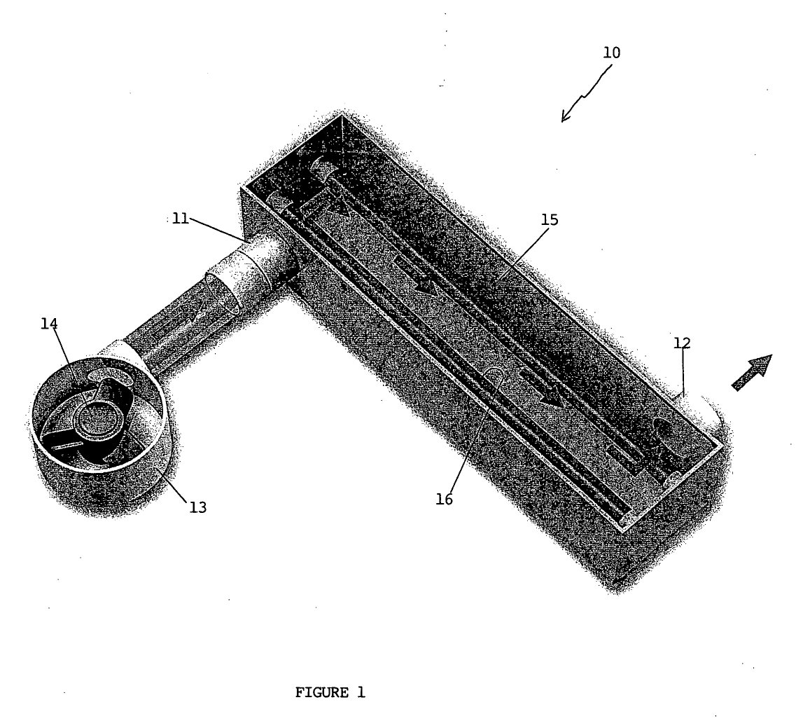

A preferred form of the present invention will now be described by way of example with reference to FIG. 1 which is a schematic perspective illustration of a UV sterilisation air-flow chamber and associated air pump.

### DESCRIPTION OF THE PREFERRED EMBODIMENT

In the accompanying drawing there is depicted schematically an airflow chamber 10. The chamber 10 is in the form of an elongate housing typically formed of plastics material having an air inlet port 11 at one end and an air outlet port 12 at the other end. There would be a lid or cover fitted to the chamber but for clarity this is not shown.

Within the chamber there is a pair of elongate ultraviolet fluorescent tubes 15 and 16 past which air flowing from the inlet port to the outlet port flows. To cause this flow, there is attached to the inlet port 11 an air pump housing 13 having a motor-driven fan 14 located therein. The motor driven fan 14 and the fluorescent tubes received electricity from an external power source (not shown).

In use the ultraviolet fluorescent tubes 15 and 16 as well as the motor driven fan 14 are activated by a switch or switches (not shown) such that air is caused to flow through the chamber as it passes the tubes and is thereby subjected to ultraviolet radiation as it flows from the inlet port 11 to the outlet port 12.

The device would typically be used to extract air from an isolated patient in an infectious disease ward of a hospital.

It should be appreciated that modifications and alterations obvious to those skilled in the art are not to be considered as beyond the scope of the present invention. For example, the airflow direction could be reversed by having the fan 14 drawn air from the chamber instead of delivered air thereto. Also, the fan might be formed integrally with the chamber. Furthermore, the chamber interior might be highly reflective to increase impingement of light on the airflow.

1. A sterilisation chamber comprising:
an elongate housing,
an air inlet port at or adjacent to one end of the housing,
an air outlet port at or adjacent to the other end of the housing, and
an elongate ultraviolet fluorescent tube mounted within the housing and extending substantially from the inlet port to the outlet port such that air passing through the housing from one end to the other encounters ultraviolet light emitted by the fluorescent tube en route from the inlet port to the outlet port.

2. The sterilisation chamber of claim 1 comprising another elongate ultraviolet fluorescent tube parallel to and coextensive with the first said tube.

3. The sterilisation chamber of claim 1 having attached to its air inlet port and/or air outlet port an air pump to cause airflow through the chamber from the inlet port to the outlet port.
